(19) United States
(12) Patent Application Publication
(10) Pub. No.: US 2025/0360214 A1
(43) Pub. Date: Nov. 27, 2025

(54) NON-TOXIC PHOTOSENSITIZER COMPOSITION AND TWO-COMPONENT TYPE PHOTOSENSITIZER COMPOSITION COMPRISING THE SAME

(71) Applicant: KOREA ATOMIC ENERGY RESEARCH INSTITUTE, Daejeon (KR)

(72) Inventor: Il Lae JUNG, Sejong-si (KR)

(21) Appl. No.: 19/215,568

(22) Filed: May 22, 2025

(30) Foreign Application Priority Data
May 23, 2024 (KR) 10-2024-0067067

(51) Int. Cl. A61K 41/00; A61K 47/02; A61K 47/10; A61K 47/20

(52) U.S. Cl. CPC A61K 41/0071; A61K 47/02; A61K 47/10; A61K 47/20

(57) ABSTRACT

The present disclosure relates to a non-toxic photosensitizer composition and a two-component type photosensitizer composition including it, and more specifically, a photosensitizer composition including a photosensitizer and a solvent for the photosensitizer including hydrochloric acid, DMSO, and water, wherein the solvent includes 1-10 vol % DMSO, 0.001 mN-50 mN hydrochloric acid, and a balance of water, based on a total volume of the photosensitizer composition; and a two-component type photosensitizer composition including a first liquid and a second liquid, wherein, when the first liquid includes 1 10 vol % DMSO and a photosensitizer, the second liquid includes 0.001 mN-50 mN of hydrochloric acid and water, and wherein, when the first liquid includes 1-10 vol % DMSO, a photosensitizer, and 0.001 mN-50 mN hydrochloric acid, based on a total volume of the photosensitizer composition, the second liquid includes water, are provided.

CROSS-REFERENCE TO RELATED APPLICATION(S)

This application claims benefit of priority to Korean Patent Application No. 10-2024-0067067 filed on May 23, 2024 in the Korean Intellectual Property Office, the disclosure of which is incorporated herein by reference in its entirety.

### BACKGROUND

### 1. Field

The present disclosure relates to a non-toxic photosensitizer composition and a two-component type photosensitizer composition including the same, and more specifically, to a photosensitizer composition capable of dissolving a photosensitizer with high solubility and dissolved in a non-toxic solvent, and a two-component type photosensitizer composition having significantly improved storage stability.

### 2. Description of Related Art

Photodynamic therapy (PDT) may be a technology for selectively destroying target cells by using a photosensitizer activated by light and a laser of a specific wavelength, and may be applied to not only treatment of anticancer but also treatments of skin diseases, antibacterial treatment, anti-inflammatory treatment, or the like, and may be a next-generation photoresponsive treatment technology that may be applied to medical devices such as a wound dressing, a microneedle, or the like. For example, photodynamic therapy for cancer may necroticize cancer cells by administering the photosensitizer to a subject, for example, via intravenous injection, waiting for a certain period in time for the photosensitizer to be transferred to tumor cells, and then irradiating the subject with red light an appropriate wavelength. In addition, the of photosensitizer may be applied in a form of a medical device, for example, in a form of a drug loaded on a wound dressing, in a form in which a spray is sprayed, or in a form applied on a microneedle or the like, to be penetrated into various skin sites of animals and humans, and may be then irradiated with red light and near-infrared light of an appropriate wavelength to treat other diseases.

The photosensitizer, which may be a key substance required for the photodynamic therapy, may be excited by light, and the excited photosensitizer may excite oxygen molecules to be converted into singlet oxygen, which directly destroys tumor tissues, or may create new radicals to selectively attack or destroy only cancer cells, various tumor tissues, or tissues or diseased sites infected with bacteria, viruses, or the like. A representative substance of this photosensitizer may be a porphyrin-based compound.

However, since porphyrin-based compounds used as photosensitizers have low water solubility, various efforts for improving water solubility have been made, but there was no clear solution. For example, 3-[(3,5-dimethyl-1H-pyrazole-4-yl)methyl]-3-devinylpyropheophorbide-a methyl ester (MPP100), which may be in the spotlight as a next-generation photosensitizer, has high activity, but extremely low water solubility, so available solvents thereof are limited to dimethylsulfoxide (DMSO), tetrahydrofuran (THF), or the like, which may be representative non-polar solvents, making it difficult to apply the photosensitizer as an innovative therapeutic agent.

Furthermore, when DMSO and THF are applied as solvents for the photosensitizer MPP100 as known, the solvents may not be only unsuitable for application in actual medical fields due to high human toxicity, but also difficult to apply to various formulations due to use of the non-polar solvents. Therefore, there may be a high difficulty in developing it as an actual therapeutic agent. In particular, since such non-polar solvents destroy lipid structures of exosomes, liposomes, nanoparticles, or the like, there may be a limitation that the photosensitizers cannot be loaded into exosomes, or liposomes, nanoparticles, or the like cannot be used as carriers.

Therefore, when a solvent having improved solubility of a photosensitizer and having non-toxicity, which may be applied to animals and humans, thereby expanding a range of application, it is expected that since solubility of the photosensitizer increases to be safely administered to patients, the photosensitizer may be used in clinical trials targeting animals and humans, and may enable loading of new types of drug carriers such as exosomes, liposomes, or the like.

### SUMMARY

An aspect of the present disclosure is to provide a non-toxic photosensitizer composition having increased solubility of a photosensitizer.

Another aspect of the present disclosure is to provide a two-component composition capable of acquiring a non-toxic photosensitizer composition having increased solubility of a photosensitizer.

According to an aspect of the present disclosure, a photosensitizer composition including a photosensitizer and a solvent for the photosensitizer including hydrochloric acid, DMSO, and water, wherein the solvent includes 1 to 10 vol % of DMSO, 0.001 mN to 50 mN of hydrochloric acid, and a balance of water, based on a total volume of the photosensitizer composition, is provided.

According to another aspect of the present disclosure, a two-component type photosensitizer composition includes a first liquid and a second liquid, wherein, when the first liquid includes 1 to 10 vol % of DMSO and a photosensitizer, based on a total volume of the photosensitizer composition, the second liquid includes 0.001 mN to 50 mN of hydrochloric acid and water, and wherein, when the first liquid includes 1 to 10 vol % of DMSO, a photosensitizer, and 0.001 mN to 50 mN of hydrochloric acid, based on a total volume of the photosensitizer composition, the second liquid includes water, is provided.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 illustrates results of confirming toxicity of each component of a photosensitizer solvent that may be included in a photosensitizer composition of the present disclosure.

FIG. 2 illustrates results of confirming solubility of MPP100 while fixing a concentration of DMSO and varying a concentration of HCl.

FIG. 3 illustrates results of confirming solubility of MPP100 while fixing a concentration of HCl and varying a concentration of DMSO.

FIG. 4 illustrates results of confirming solubility of MPP100 according to various compositions of a photosensitizer composition.

FIG. 5 illustrates results of confirming solubility of MPP100 of a solvent having a composition of Inventive Example 2 according to a concentration of MPP100.

FIG. 6 illustrates results of performing an L-929 cytotoxicity test based on an MFDS guideline to confirm cytotoxicity of a photosensitizer composition.

FIG. 7 illustrates results of confirming stability of a photosensitizer composition over time in various compositions of the photosensitizer composition.

FIG. 8 illustrates results of confirming an improvement in stability of a photosensitizer composition when manufactured in a two-component form.

FIG. 9 illustrates the difference in stability when a photosensitizer composition of Inventive Example 3 may be manufactured in a one-component form and a two-component form.

### DETAILED DESCRIPTION

Hereinafter, preferred embodiments of the present disclosure will be described with reference to the attached drawings. However, embodiments of the present disclosure may be modified in various other forms, and the scope of the present disclosure is not limited to embodiments described below.

According to the present disclosure, a photosensitizer composition using a solvent capable of dissolving a non-polar photosensitizer having high solubility may be provided, and the solvent of the present disclosure may be further non-toxic.

More specifically, a photosensitizer composition of the present disclosure may include a photosensitizer and a solvent for the photosensitizer including hydrochloric acid, DMSO, and water, wherein the solvent includes 1 to 10 vol % of DMSO, 0.001 mN or more and 50 mN or less of hydrochloric acid, and a balance of water, based on a total volume of the photosensitizer composition.

The water that may be used in the present disclosure is not particularly limited, and may include distilled water (DW), other purified water, or the like.

In this case, the photosensitizer of the present disclosure is not particularly limited as long as it is non-polar and has low water solubility, but for example, the photosensitizer that may be applied to the present disclosure may be porphyrin, bacteriochlorin, phthalocyanine, a photosensitizing cyclic tetrapyrrole, a 5-aminolevulinic acid derivative, or the like, and preferably, a porphyrin compound having a methyl functional group.

The porphyrins compound may include at least one selected from the group consisting of, for example, hematoporphyrin, protoporphyrin IX, talaporfin, pheophorbide A, and chlorin e6, and may be, for example, (3-[(3,5-dimethyl-1H-pyrazole-4-yl)methyl]-3-devinylpyropheophorbide-a methyl ester) (MPP100) of the following chemical formula (1) or methyl pyropheophorbide-a (MPPa) of the following chemical formula (2).

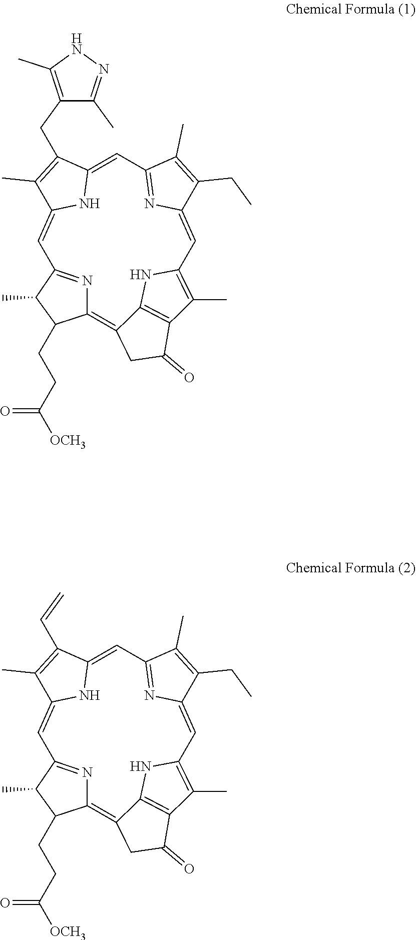

In the present disclosure, the solvent for dissolving the photosensitizer may include hydrochloric acid and a DMSO aqueous solution, wherein the hydrochloric acid may be 0.001 mN or more and 50 mN or less of hydrochloric acid, and may be included in a concentration of for example, 0.1 mN to 25 mN, 1 mN or more and 25 mN or less, or less than 25 mN, preferably 0.25 mN to 10 mN.

When the hydrochloric acid is included in a concentration less than the concentration ranges mentioned above, solubility of the photosensitizer may decrease, and when the hydrochloric acid is included in a concentration exceeding 50 mN, precipitation of the photosensitizer may occur.

DMSO may be a toxic substance reported to have excellent solubility in non-polar substances but cause various cell damages, and according to the present disclosure, such DMSO may be included in an amount of 1 to 10 vol % based on a total volume of the photosensitizer composition. For example, DMSO may be included in an amount of 5 to 7.5 vol %. However, when DMSO exceeds 10 vol % based on a total volume of the photosensitizer composition, toxicity of the photosensitizer composition may entirely increase and solubility of the photosensitizer may entirely decrease, and when DMSO is used at less than 1 vol %, solubility of the photosensitizer decreases or there may be a high risk of precipitation occurring over time.

The photosensitizer composition of the present disclosure may include the photosensitizer in an amount of more than 0 to 5 mg per 1 mL of the photosensitizer composition, for example, in an amount of 0.001 to 2 mg per 1 mL of the photosensitizer composition. Although not limited to this amount, when photosensitizer is included in an amount exceeding 5 mg per 1 mL of the photosensitizer composition, solubility of the photosensitizer may decrease.

Furthermore, it may not be excluded that the photosensitizer composition of the present disclosure additionally includes polyethylene glycol (PEG). However, when the solvent of the present disclosure additionally includes polyethylene glycol (PEG, average molecular weight 300), an amount of polyethylene glycol (PEG) included in this case may be 20 vol % or less based on a total volume of the photosensitizer composition, and may be, for example, 15 vol % or less based on a total volume of the photosensitizer composition. When an amount of polyethylene glycol (PEG) exceeds 20 vol %, toxicity of the photosensitizer composition may increase.

Furthermore, the photosensitizer composition of the present disclosure may be provided in a two-component form to increase dissolution stability. All of the above-described contents related to the photosensitizer composition may be equally applied to a two-component type photosensitizer composition below.

For example, a two-component type photosensitizer composition of the present disclosure may include a first liquid and a second liquid, and the first liquid and the second liquid may be prepared separately, and when the first liquid includes 1 to 10 vol % of DMSO and a photosensitizer, based on a total volume of the photosensitizer composition, the second liquid may include 0.001 mN to 50 mN of hydrochloric acid and water, and when the first liquid includes 1 to 10 vol % of DMSO, a photosensitizer, and 0.001 mN to 50 mN of hydrochloric acid, based on a total volume of the photosensitizer composition, the second liquid may include water.

More specifically, a two-component type photosensitizer composition of the present disclosure may include a first liquid including 1 to 10 vol % of DMSO and a balance of a photosensitizer, based on a total volume of the photosensitizer composition; and a second liquid including 0.001 mN to 50 mN of hydrochloric acid and water, based on a total volume of the photosensitizer composition, wherein the first liquid and the second liquid may be prepared separately.

Alternatively, the two-component type photosensitizer composition of the present disclosure may include a first liquid including 1 to 10 vol % of DMSO, a photosensitizer, and 0.001 mN or more and 50 mN or less of hydrochloric acid, based on a total volume of the photosensitizer composition; and a second liquid including water, wherein the first liquid and the second liquid may be manufactured separately. In this case, DMSO and hydrochloric acid of the first liquid may include, for example, 1 to 7.5 vol % of DMSO and 0.001 mN or more and 10 mN or less of hydrochloric acid, based on a total volume of the photosensitizer composition, and preferably, 1 to 5 vol % of DMSO and 0.001 mN or more and 5 mN or less of hydrochloric acid, based on a total volume of the photosensitizer composition.

Preferably, when an amount of hydrochloric acid is 10 mN or more, the first liquid may not include water and may include only a photosensitizer dissolved in 100% DMSO, or may include a photosensitizer and hydrochloric acid dissolved in 100% DMSO, and the second liquid may include hydrochloric acid and water or may include only water, and the two liquids may be mixed immediately before use to prepare a photosensitizer for a final usage, for example, a clinical usage.

Solubility of the photosensitizer in the photosensitizer composition of the present disclosure may be affected by a composition of each of the first liquid and the second liquid in the two-component type photosensitizer composition.

When prepared in a two-component form, the first liquid may additionally include more than 0 to 15 vol % of polyethylene glycol (PEG) based on a total volume of the photosensitizer composition.

For example, to stably maintain solubility of the photosensitizer, which may be a non-polar substance having a very high hydrophobicity, when an amount of hydrochloric acid is 10 mN or more, in preparation of the photosensitizer composition of the present disclosure, a mixture of the photosensitizer and DMSO, or a mixture of the photosensitizer, DMSO, and hydrochloric acid may be prepared as a first liquid. Separately, a hydrochloric acid aqueous solution including hydrochloric acid and water, or a solution including only water may be prepared as a second liquid. In a case in which 1 to 7.5 vol % of DMSO is included based on a total volume of the photosensitizer composition and an amount of hydrochloric acid is 10 mN or less, the first liquid may be prepared to include DMSO, a photosensitizer, and hydrochloric acid.

The first liquid and the second liquid may be mixed at an appropriate time before use, and are preferably mixed within 24 hours before use, for example, immediately before use.

When compositions of the first liquid and the second liquid of the photosensitizer composition suggested by the present disclosure are different during the preparation thereof, solubility of the photosensitizer may be insufficient.

Hereinafter, the present disclosure will be described in more detail through specific examples. The following examples are merely illustrative to help understand the present disclosure, and the scope of the present disclosure is not limited thereto.

### EXAMPLES

### 1. Selection of Photosensitizer

For preparation of a photosensitizer composition, MPP100 represented by the following chemical formula (1) was obtained from Dr. I&B Co., Ltd. and used as a photosensitizer, respectively.

3-[(3, 5-dimethyl-1H-pyrazole-4-yl)methyl]-3-devinylpyropheophorbide-a methyl ester (MPP100)

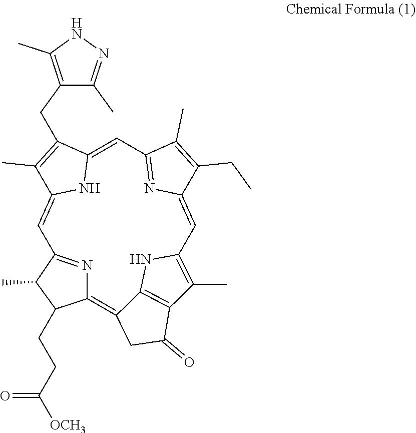 [SMILES: CCC1=C(C)c2cc3[nH]c(cc4nc(c5c6[nH]c(cc1n2)c(C)c6C(=O)C5)C(CCC(=O)OC)[C@@H]4C)c(C)c3Cc1c(C)n[nH]c1C]

In a compound of chemical formula (1), a molecular formula may be C₃₈H₄₂N₆O₃ and a molecular weight may be 630.7785.

### 2. Confirmation of Toxicity of Each Component included in Solvent Composition for Photosensitizer

A cytotoxicity test was performed on PEG, DMSO, and HCl, which may be used as components of a solvent composition for photosensitizer of the present disclosure, according to the following Experimental Method 1.

### Experimental Method 1

RAW264.7 (5×10\hat{ }5 cells/well in 6 well plate) was used as a cell line, and 1XDMEM (supplemented with 7% FBS and 1% antibiotics) medium (Thermo fisher scientific, 11995-065) was used.

Raw264.7 cells, which may be animal model cells, were seeded into 6 well plates containing 2 mL of DMEM medium to have an initial cell number of 5×10⁵ cells/well, and then cultured in a CO₂ incubator at 37° C. for 1 day under CO₂ conditions. After 1 day of culture, solvent composition samples were added and cultured for an additional 2 days under the same conditions, respectively. After that, MTT assay was performed to confirm cytotoxicity.

In this case, the MTT assay method using an MTT assay kit (EZ-CYTOX-company, Dogenbio-model number: EZ-500) first prepared 5×10\hat{ }5/well (total vol. 2 ml) in a 6-well plate for seeding, and then cultured for 24 h under 37° C., 5% CO₂ conditions. A blank (only media) without cells was also prepared at the same time. An experimental material was treated according to conditions, and then cultured and reacted for 24 h under 37° C./5% CO₂ conditions. Additionally, 200 μL Ez-cytox ( 1/10) was added to each of the wells, and reacted for 1 h. The wells were shaken gently before measuring absorbance, 200 μL thereof was then transferred to a 96-well plate, the absorbance was measured at a wavelength of 450 nm, and a viability rate was calculated according to the following equation (1).

$$Viability ( \% ) : ( ( Sample - blank ) / ( Control - blank ) ) ★100 \qquad Equation ( 1 )$$

A cytotoxicity test was performed on PEG, DMSO, and HCl according to the experimental method.

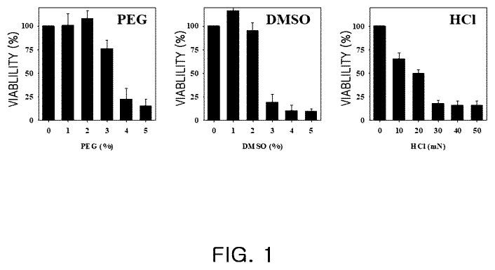

As a result, referring to FIG. 1, no toxicity was observed at 3 vol % or less of PEG, 2 vol % or less of DMSO, and 10 mN or less of HCl. According to an MEDS medical device guideline for cytotoxicity testing of a medical device, a test subject material was diluted by ⅕ and then the cytotoxicity test was performed, and when the test subject material illustrates 50% or more cytotoxicity, the medical device cannot be approved. Therefore, when result values derived from FIG. 1 (PEG 3 vol % or less, DMSO 2 vol % or less, HCl 10 mN) are multiplied by 5, a non-toxic limit may be obtained. In conclusion, the non-toxic limit was PEG 3 vol % or less×5=15 vol %, DMSO 2 vol % or less×2=10 vol %, and HCl 10 mN×5=50 mN.

In conclusion, referring to FIG. 1, it was found that concentrations of DMSO, HCl, and PEG in a solvent for a non-toxic photosensitizer should be DMSO 10 vol % or less, HCl 50 mN or less, and PEG 15 vol % or less, respectively, based on a volume of a photosensitizer composition. When diluted to ⅕, since actual final concentrations applied to the cell were 2 vol % of DMSO, 10 mN of HCl, and 3 vol % of PEG, it may be said to be a suitable solvent because no cytotoxicity was observed at this concentration, as illustrated in FIG. 1.

### 3. Photosensitizer Solubilization Experiment of Solvent Composition for Photosensitizer

According to the results of the toxicity test, an aqueous solution including concentrations of 10 vol % or less of DMSO and 50 mN or less of HCl as a photosensitizer solvent composition was prepared to test solubilization possibility of the MPP100 photosensitizer.

To this end, DMSO and HCl were combined while fixing an amount of one component of DMSO or HCl and changing an amount of the other component thereamong, and then 500 μg of MPP100, which may be an exemplary solubilization target material, was dissolved. In this case, a volume of a final sample including the photosensitizer, DMSO, hydrochloric acid, and water was fixed to a total of 1 mL, i.e., an final concentration of MPP100 corresponded to 500 μg/mL of MPP100.

In this case, first, 1N HCl and DW were sequentially added to a 50 μL mixture of MPP100 and DMSO (corresponding to 500 μg of MPP100) in which MPP100 was dissolved in DMSO (100%) in an amount of 10 mg/mL, to make a final volume 0 of 1 mL, and then, after standing at room temperature for 1 hour, absorbance was measured at 405 nm to calculate solubility of MPP100. Samples manufactured according to the present disclosure may be collectively referred to as “JL buffer.”

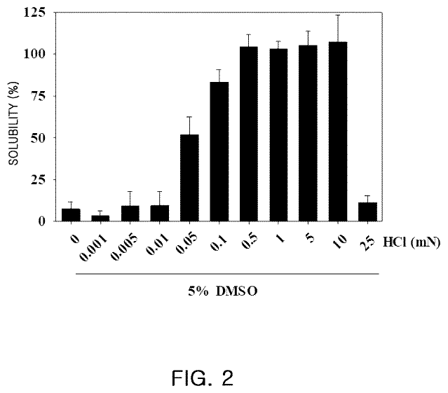

As a result, as illustrated in FIG. 2, in tests in which 5% DMSO was fixed and a concentration of HCl was varied to 0.001, 0.005, 0.01, 0.05, 0.1, 0.5, 1, 5, 10, and 25 mN, when 25 mN HCl was added, precipitation occurred, but when 0.5 to 10 mN HCl were used, solubility of MPP100 was 100%, confirming that it could be completely solubilized.

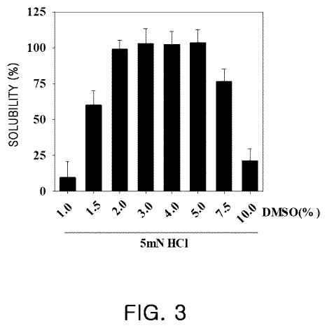

Under an HCl 5 mN fixed condition, the tests were performed such that final DMSO concentrations were 1.0, 1.5, 2.0, 3.0, 4.0, 5.0, 7.5, and 10 vol %, by a process in which final DMSO concentrations were adjusted by additionally adding 100% DMSO to have 1 to 10 volt, in a state in which 10 μL of a solution including 50 mg/mL of MPP dissolved in 100% DMSO (MPP 50 mg/mL in 100% DMSO) was basically prepared. In this case, concentrations of MPP100 in the tests for each DMSO concentration were all 500 μg/mL. After leaving it at room temperature for 1 hour, absorbance was measured at 405 nm to calculate solubility of MPP100. When concentrations of DMSO were changed, as can be seen in FIG. 3, it was hardly dissolved in 10 vol % DMSO, but when 2 to 5 vol % DMSO were applied, it showed 100% solubility.

As a result, it was confirmed that solubility was not improved when concentrations of components constituting the solvent were high, but that an appropriate ratio of components was important.

### 4. Preparation of Photosensitizer Composition

Based on the experimental results as above, a non-toxic MPP100 solubilization buffer (hereinafter, referred to as ‘JL buffer’) was mixed with 500 μg of photosensitizer MPP100 using compositions, as illustrated in Table 1, as solvents, and solubility was measured and illustrated in Table 1. In this case, a final volume after preparation was 1 mL. In Table 1 below, DW means distilled water.

TABLE 1

| | | MPP Solution | 100% | Final | 1N | DW | Final | |
|---|---|---|---|---|---|---|---|---|
| | | (μL) in 100% | DMSO | DMSO | HCl | (Water) | HCl | Solubility |
| Examples | Samples | DMSO | (μL) | (vol % ) | ( μL) | (μL) | (mN) | (%) |
| Comparative | DMSO 5% | 50 | — | 5 | — | 950 | 0 | ≤10 |
| Ex. 1 | | | | | | | | |
| Inventive | DMSO 5%/HCl | 50 | — | 5 | 5 | 945 | 5 | 100% |
| Ex. 1 | 5 mN | | | | | | | |
| Inventive | DMSO 5%/HCl | 50 | — | 5 | 10 | 940 | 10 | 100% |
| Ex. 2 | 10 mN | | | | | | | |
| Inventive | DMSO 7.5%/HCl | 50 | 25 | 7.5 | 10 | 915 | 10 | ≤80 |
| Ex. 3 | 10 mN | | | | | | | |
| Comparative | DMSO 5%/HCl | 50 | — | 5 | 25 | 925 | 25 | ≤10 |
| Ex. 2 | 25 mN | | | | | | | |
| Comparative | DMSO 7.5%/HCl | 50 | 25 | 7.5 | 25 | 900 | 25 | ≤10 |
| Ex. 3 | 25 mN | | | | | | | |
| Comparative | JH ( 40/0.5) | 50 | 350 | 40 | 500 | 100 | 500 | ≤10 |
| Ex. 4 | | | | | | | | |
| Comparative | 1 (MD) 2 ( HCl DW) | 50 | 25 | 7.5 | 25 | 900 | 25 | ≤10 |
| Ex. 5 | | | | | | | | |
| Comparative | 1 (MDHCl) 2 ( DW) | 50 | 25 | 7.5 | 25 | 900 | 25 | ≤10 |
| Ex. 6 | | | | | | | | |
| Inventive | 1 (MDHCl) 2 ( DW) | 50 | — | 5 | 0.25 | 949.75 | 0.25 | 100% |
| Ex. 4 | | | | | | | | |
| Inventive | 1 (MDHCl) 2 ( DW) | 50 | — | 5 | 2.5 | 947.5 | 2.5 | 100% |
| Ex. 5 | | | | | | | | |

*MPP solution (μL) in 100% DMSO represents an amount of solution in which MPP100 was dissolved in 100% DMSO at a concentration of 10 mg/mL (50 μL was supplied to obtain a final concentration of 500 μg/mL).

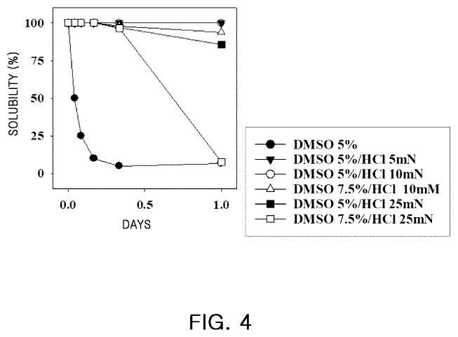

According to Table 1, in Comparative Example 1 including 5% DMSO and water without addition of hydrochloric acid, it was confirmed that precipitation occurred immediately. When treated with 5 volume % DMSO, in both Inventive Examples 1 and 2 including 5 mM HCl and 10 mM HCl, respectively, solubility of MPP100 was stably maintained for at least 1 day without precipitation, and in Inventive Example 3 having a HCl concentration of 10 mM when treated with 7.5 volume % DMSO, solubility was maintained relatively stable, while in Comparative Examples 2 and 3, when 25 mM HCl was treated, solubility decreased over time and precipitation occurred (FIG. 4).

From the results, it can be confirmed that an appropriate combination of DMSO and HCl is necessary to solubilize MPP100.

### 5. Confirmation of Photosensitizer Solubility of Photosensitizer Composition

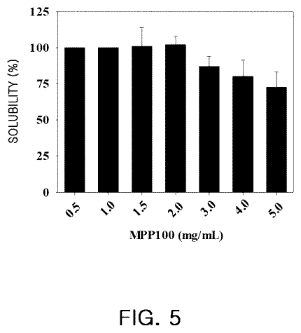

Solubility of MPP100 in a solvent having the composition of Inventive Example 1 was investigated according to a concentration of the MPP100. In this case, the concentration of the MPP100 was adjusted to 0.5, 1.0, 1.5, 2.0, 3.0, 4.0, and 5.0 mg/mL to prepare samples, and after standing at room temperature for 1 hour, absorbance was measured at 405 nm to calculate solubility of MPP100. As a result, as can be confirmed in FIG. 5, MPP100 could be stably dissolved at 100% solubility up to 2 mg/mL (FIG. 5). In this case, 50 mg/mL stock of MPP100 was used. Solubility of high concentration MPP100 (3 to 5 mg/mL) that did not show 100% solubility in FIG. 5 may increase further when a stock concentration of MPP100 increased.

### 6. Confirmation of Cytotoxicity of Photosensitizer Composition

To confirm cytotoxicity of the photosensitizer composition of the present disclosure, an L-929 cytotoxicity test based on the MFDS guideline was performed.

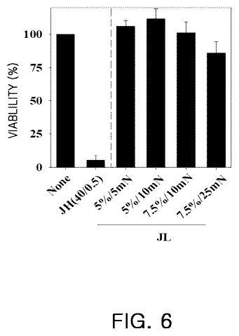

As a result, as can be confirmed in FIG. 6, while Comparative Example 4 showed severe cytotoxicity, when the solvent belonging to an example of the present disclosure was used, no cytotoxicity was observed at all, confirming that it was a novel non-toxic buffer suitable for medical device approval.

As such, a solvent for photosensitizer of the present disclosure may completely solubilize 2 mg/mL of MPP100 without any cytotoxicity. In particular, a solvent for photosensitizer of the present disclosure showed no cytotoxicity at ⅕ of a concentration required for the MFDS guideline test.

### 7. Confirmation of Stability of Photosensitizer Composition

Long-term stability of a photosensitizer composition including MPP100 (500 μg/mL) was investigated.

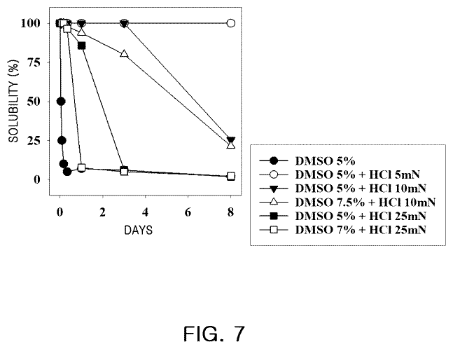

As can be seen in FIG. 7, 100% solubility was generally observed initially, but precipitation was observed in some compositions over time, except for the composition of Inventive Example 1. A degree of precipitation of such precipitates tended to increase, as concentrations of DMSO and HCl increased, and this result was a factor that hinders stable distribution in the market in the long term.

Therefore, to ensure long-term stability, the photosensitizer composition of the present disclosure was manufactured in a two-component formulation using a composition of Comparative Example 5, which has a high concentration, as an example (Comparative Example 5 and Comparative Example 6). In this case, Comparative Example 5 was sealed by adding only MPP100 and DMSO to a first liquid, and HCl and DW to a second liquid, and Comparative Example 6, Inventive Examples 4 and 5 were sealed by adding only MPP100, DMSO, and HCl to a first liquid, and DW to a second liquid.

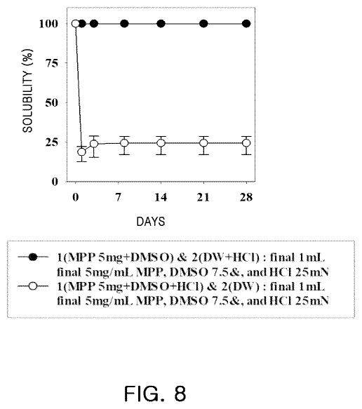

In this case of manufacturing in a two-component form, as can be confirmed in FIG. 8, precipitation occurred immediately in Comparative Example 6, but it was confirmed that precipitation did not occur for 28 days or more in Comparative Example 5.

Referring to the results of Inventive Examples 4 and 5, when a concentration of DMSO and HCl was adjusted, especially when concentrations of DMSO and HCl were low at 1 to 10 volt based on a total volume of the photosensitizer composition and 100% solubility may be secured even when mixing MPP100, DMSO, and HCl in a first liquid and including DW in a second liquid.

Therefore, when the first and second liquids were mixed at the time when use is required after manufacturing in a two-component formulation, storage stability may be significantly improved, but a stability improvement effect may vary depending on the composition and concentration of each formulation.

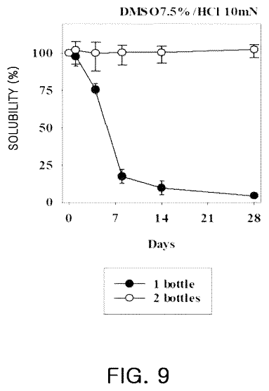

When Inventive Example 3 of Table 1 was manufactured in a two-component form in the same manner as Comparative Example 5, it was found that no precipitation occurred for 28 days (FIG. 9). In FIG. 9, ‘1 bottle’ indicates that a photosensitizer composition of Inventive Example 3 was stored in one container, and ‘2 bottles’ indicates that the photosensitizer composition of Inventive Example 3 was manufactured in a two-component form in the same manner as Comparative Example 5. In this way, compared to the existing one-component formulation, the two-component formulation has an advantage of allowing long-term stable storage of MPP100 without precipitation even when concentrations of DMSO and HCl were high.

As a result of this experiment, it was found that to secure solubility of the photosensitizer in the present disclosure, maximum solubility was secured when DMSO was mixed first with MPP100, followed by HCl and water, and preferably water was mixed last.

When adding PEG, the treatment time was preferably before or immediately after DMSO was treated.

Therefore, it is preferable to prepare a solution by first dissolving MPP 100 powder in DMSO and then mixing it with the remaining components (HCl and water) rather than directly dissolving MPP 100 powder in a solution already mixed with other solvents in terms of securing solubility of MPP100.

A photosensitizer composition of the present disclosure may include a non-toxic solvent of the present disclosure having a new composition to have high solubility for a photosensitizer while having non-toxicity, and thus may be used in clinical trials targeting animals and humans, and may be utilized in development of various formulations, to be expected to be widely applied in related fields. In addition, a photosensitizer composition of the present disclosure may be manufactured in a two-component form, and may significantly improve storage stability.

While example embodiments have been illustrated and described above, it will be apparent to those skilled in the art that modifications and variations could be made without departing from the scope of the present disclosure as defined by the appended claims.

What is claimed is:

1. A photosensitizer composition comprising:
a photosensitizer; and
a solvent for the photosensitizer including hydrochloric acid, DMSO, and water,
wherein the solvent includes 1 to 10 vol % of DMSO, 0.001 mN to 50 mN of hydrochloric acid, and a balance of water, based on a total volume of the photosensitizer composition.

2. The photosensitizer composition of claim 1, wherein the photosensitizer is a porphyrin compound having a carboxyl functional group.

3. The photosensitizer composition of claim 2, wherein the porphyrin compound includes at least one selected from the group consisting of hematoporphyrin, protoporphyrin IX, talaporfin, pheophorbide A, and chlorin e6.

4. The photosensitizer composition of claim 1, wherein the photosensitizer is included in an amount of more than 0 to 5 mg per 1 mL of the photosensitizer composition.

5. The photosensitizer composition of claim 1, further including more than 0 to 20 vol % of polyethylene glycol (PEG) based on a total volume of the photosensitizer composition.

6. A two-component type photosensitizer composition comprising:
a first liquid and a second liquid,
wherein the first liquid and the second liquid are prepared separately,
wherein, when the first liquid includes 1 to 10 vol % of DMSO and a photosensitizer, based on a total volume of the photosensitizer composition, the second liquid includes 0.001 mN to 50 mN of hydrochloric acid and water, and
wherein, when the first liquid includes 1 to 10 vol % of DMSO, a photosensitizer, and 0.001 mN to 50 mN of hydrochloric acid, based on a total volume of the photosensitizer composition, the second liquid includes water.

7. The two-component type photosensitizer composition of claim 6, wherein the photosensitizer is a porphyrin compound having a carboxyl functional group.

8. The two-component type photosensitizer composition of claim 7, wherein the porphyrin compound includes at least one selected from the group consisting of hematoporphyrin, protoporphyrin IX, talaporfin, pheophorbide A, and chlorin e6.

9. The two-component type photosensitizer composition of claim 6, wherein the photosensitizer is included in an amount of more than 0 to 5 mg per 1 mL of the photosensitizer composition.

10. The two-component type photosensitizer composition of claim 6, wherein the first liquid further includes more than 0 to 20 vol % of polyethylene glycol (PEG) based on a total volume of the photosensitizer composition.
